(19) United States
(12) Patent Application Publication
(10) Pub. No.: US 2010/0248342 A1
(43) Pub. Date: Sep. 30, 2010

(54) TIP DRIVE APPARATUS AND CANTILEVER TIP

(75) Inventors: Kiyohiko TATEYAMA, Sagamihara-shi (JP); Yasuo Sasaki, Machida-shi (JP); Yuka Imaoka, Hino-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/813,077

(22) Filed: Jun. 10, 2010

Related U.S. Application Data
(63) Continuation of application No. PCT/JP2008/072199, filed on Dec. 5, 2008.

(30) Foreign Application Priority Data
Dec. 27, 2007 (JP) 2007-338367

(51) Int. Cl. C12M 1/00

(52) U.S. Cl. 435/285.1

(57) ABSTRACT

A tip drive apparatus is capable to moving a tip unit toward an object, while holding the tip unit at a prescribed angle. The tip unit is formed on a support unit having flexibility and directed to the object at the prescribed angle. The tip unit includes a contact side in a cross section including the distal end of the support unit and extending in the direction the support unit extends, the contact side configured to contact, at one part at least, the object, and to apply a prescribed pressure to the object. The tip unit further includes a first region including the contact side, and a second region continuous to the support unit.

### CROSS REFERENCE TO RELATED APPLICATIONS

This is a Continuation Application of PCT Application No. PCT/JP2008/072199, filed Dec. 5, 2008, which was published under PCT Article 21(2) in Japanese.

This application is based upon and claims the benefit of priority from prior Japanese Patent Application No. 2007-338367, filed Dec. 27, 2007, the entire contents of which are incorporated herein by reference.

### BACKGROUND OF THE INVENTION

1. Field of the Invention

The present invention relates to a tip drive apparatus that can move a tip unit formed on a support unit having flexibility and arranged at a prescribed angle to an object, while holding the tip unit at the prescribed angle.

2. Description of the Related Art

WO 04/092369 discloses a microinjection method and apparatus that are designed to introduce a substance, such as genes, into cell. In the technique disclosed, the substance is electrically adsorbed to the distal end of a microneedle, which is a tip unit, and the microneedle is then inserted into a cell. A pulse voltage is applied to the microneedle, moving the substance from the distal end of the microneedle and introducing the substance into the cell. The microneedle is thrust into the cell as it is minutely moved by using a piezoelectric element that can expand and contract coaxially with the microneedle. This technique is to hold genes at the distal end of the microneedle, can introduce the genes into the cell in a low-invasive manner, increasing the survival rate of the cell.

However, the microneedle formed at distal end of a cantilever tip cannot penetrate the cell membrane in some cases. This is because the microneedle invades the cell at a very small volume and also because the cell membrane has fluidity. Even if the cantilever tip is moved to such a position where its distal end may penetrate the cell membrane, the cell membrane covers up the surface of the distal end, disabling the needle from piercing the cell membrane in some cases. Consequently, the tip cannot be stably driven and the substance cannot be introduced at a sufficient rate.

The technique disclosed in the above-identified document cannot utilize a tip drive apparatus that supplies an electric current to the microneedle in order to give an electrical stimulus to a living cell so that the cell may be observed in a living state with high efficiency.

This invention has been made in view of the foregoing. An object of the invention is to provide a tip drive apparatus and a cantilever tip, which can introduce a substance into a cell at in a low-invasive manner, thus maintaining the cell at a high survival rate or apply an electrical stimulus to the cell in order to observe the living cell with high efficiency.

### BRIEF SUMMARY OF THE INVENTION

According to an aspect of embodiments, there is provided a tip drive apparatus capable to moving a tip unit toward an object, while holding the tip unit at a prescribed angle, the tip unit being formed on a support unit having flexibility and directed to the object at the prescribed angle, the tip unit includes:

a contact side in a cross section including the distal end of the support unit and extending in the direction the support unit extends, the contact side configured to contact, at one part at least, the object, and to apply a prescribed pressure to the object;

a first region including the contact side; and

a second region continuous to the support unit.

According to an another aspect of embodiments, there is provided a cantilever tip comprising:

a support unit having flexibility; and

a tip unit formed at a prescribed angle to the support unit, and configured to be attached, via a particular member, to a tip drive apparatus capable of moving the tip unit in a predetermined direction,

wherein the tip unit includes:

a contact side in a cross section including the distal end of the support unit and extending in the direction the support unit extends, the contact side configured to contact, at one part at least, an object, and to apply a prescribed pressure to the object;

a first region including the contact side; and

a second region continuous to the support unit.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing the overall configuration of a tip drive apparatus according to a first embodiment of this invention.

FIG. 2 is a diagram showing the characterizing section of the tip drive apparatus according to the first embodiment.

FIG. 3 is a diagram showing the configuration of a needle.

FIG. 4 is a side view showing the configuration of a cantilever tip having a sharpened tip unit for use in an ordinary tip drive apparatus.

FIG. 5 is a perspective view showing the configuration of the tip unit used in the tip drive apparatus according to the first embodiment.

FIG. 6 is a side view showing the configuration of the tip unit.

FIG. 7 is a magnified perspective view of the tip unit used in the first embodiment.

FIG. 8 is a diagram explaining the interference that may be caused, depending on the angle of the needle.

FIG. 9 is a diagram explaining the region in which the needle can move.

FIG. 10 is a block diagram showing the electrical configuration of the tip drive apparatus according to the first embodiment.

FIG. 11 is a flowchart explaining a tip driving method using the tip drive apparatus according to the first embodiment.

FIG. 12 is a diagram explaining how a tip is introduced by using a conventional, sharp tip unit.

FIG. 13 is a diagram explaining how a tip is introduced by using the tip unit according to the first embodiment.

FIG. 14 is a diagram presenting a microscope image of HelaS3 cells into which tip drive apparatus according to the first embodiment has introduced genes to express GFP fluorescent protein.

FIG. 15 is a diagram presenting an image of the HelaS3 cells, acquired through a phase contrast microscope, 24 hours after the genes had been introduced into the cell to express GFP fluorescent protein.

FIG. 16 is a diagram presenting a microscope image of the HelaS3 cells, acquired 24 hours after the genes had been introduced into the cell to express GFP fluorescent protein.

FIG. 17 is a perspective view showing the shape of the tip unit of a tip drive apparatus according to a second embodiment of this invention.

FIG. 18 is a side view showing the shape of the tip unit used in the second embodiment.

FIG. 19 is a diagram explaining a method of manufacturing the tip unit used in the second embodiment.

FIG. 20 is a diagram explaining how a tip is introduced by using the tip unit according to the second embodiment.

### DETAILED DESCRIPTION OF THE INVENTION

Some of the best modes for carrying out this invention will be described, with reference to the accompanying drawings.

### First Embodiment

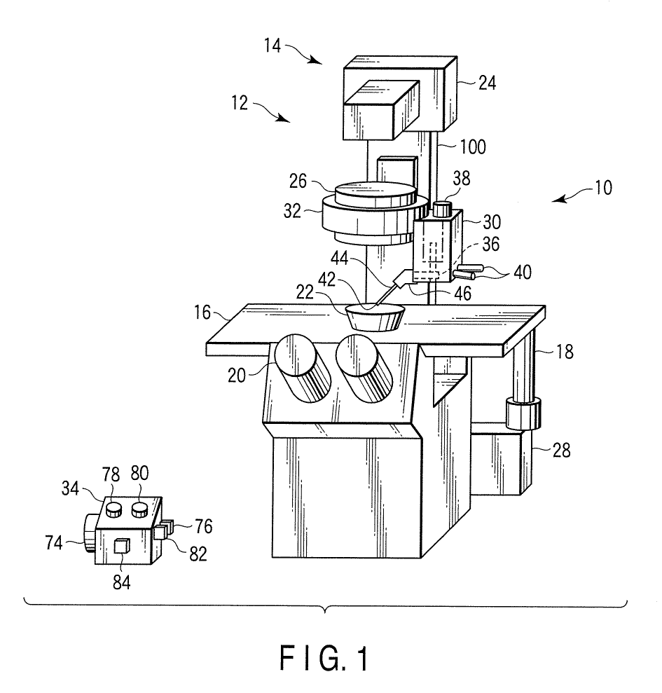

As shown in FIG. 1, a tip drive apparatus 10 according to a first embodiment of this invention is attached to an inverted microscope 12 through which to observe cells. So attached, the tip drive apparatus 10 is used.

The inverted microscope 12 has an illumination device 14, a microscope XY stage 16, a microscope XY stage handle 18, an objective lens (not shown), and an eyepiece 20. The illumination device 14 illuminates the cells set in on a dish 22. The microscope XY stage 16 moves the dish 22 in X direction and Y direction. When operated, the microscope XY stage handle 18 drives the microscope XY stage 16. The objective lens and the eyepiece 20 constitute an optical system through which to observe the light reflected from, or passing through, the cells mounted on the dish 22, or the fluorescent light emanating from the cells. At least the bottom of the dish 22 is made of transparent material such as glass, so that the cells may be observed.

The inverted microscope 12, which is a manually operable type, may be replaced by an electrically-driven type, in which the XY stage 16 is driven and controlled by a computer. Further, the inverted microscope 12 may instead be a type that has a CCD camera and can display images on a monitor.

The illumination device 14 has an illumination light source 24, a condenser lens 26, and an epi-illumination light source 28. The illumination light source 24 applies illumination light to the cells mounted on the dish 22, from the side opposite to the eyepiece 20. The condenser lens 26 receives the illumination light emitted from the illumination light source 24 and converges the light onto the cells. The epi-illumination light source 28 applies illumination light to the cells mounted on the dish 22, from the same side as the eyepiece 20.

The tip drive apparatus 10 according to this embodiment is composed of a main unit 30, a microscope adaptor 32, and an operation module 34. The microscope adaptor 32 is a unit that is attached to the condenser lens 26 of the main unit 30. As seen from FIG. 1, the main unit 30 is attached at the right side of the condenser lens 26, in front of the inverted microscope 12, where the eyepiece 20 is arranged. The operation module 34 is connected by a cable (not shown) to the main unit 30 and can be set at any desired position.

The main unit 30 has an adaptor holder unit 36, a Z-drive unit 38, and a needle-tip XY adjustment knob 40. A tip unit 42, which should be driven, is secured to a needle 44. The needle 44, which has the tip unit 42, is attached to the adaptor 46. The adaptor 46 holding the needle 44 is attached to the adaptor holder unit 36. As the adaptor holder unit 36 is moved in the Z-direction, the Z-drive unit 38 drives the tip unit 42 in the Z-direction. The needle-tip XY adjustment knob 40 moves the adaptor holder unit 36 in X direction and Y direction, adjusting the XY position of the tip unit 42.

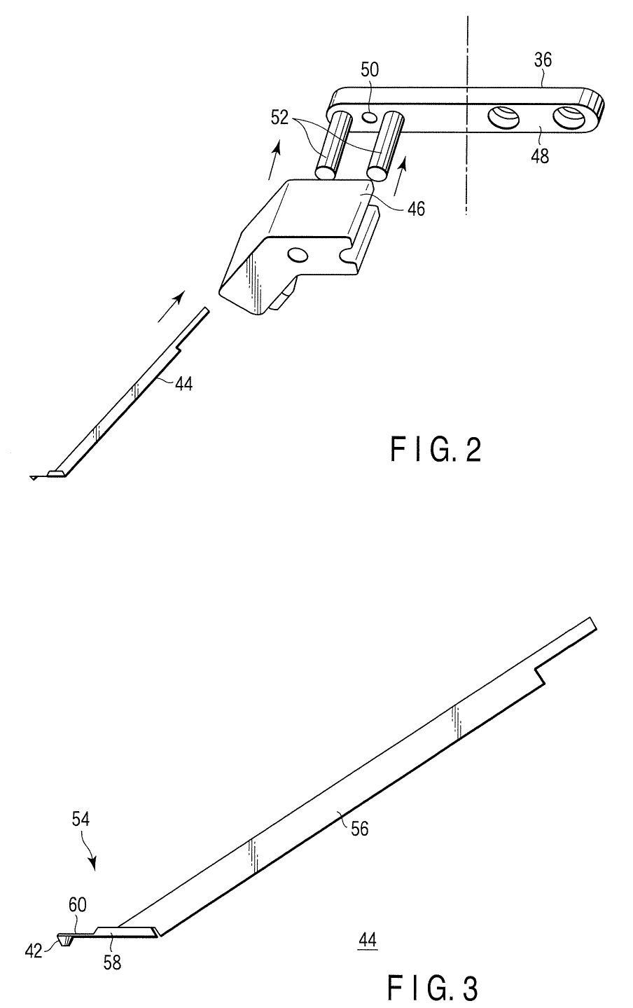

As shown in FIG. 2, the adaptor holder unit 36 has a Z-axis drive holder unit 48 configured to secure it to a linear movement mechanism (not shown) of the Z-drive unit 38 through a XY drive mechanism (not shown) (the needle-tip XY adjustment knob 40 drives the adaptor holder unit 36, in cooperation with the drive mechanism). Moreover, the adaptor holder unit 36 has an attachment member on the side opposite to the Z-axis drive holder unit 48 in the lengthwise direction. The attachment member is configured to attach the adaptor 46 to, and detached the same from, the adaptor holder unit 36. The attachment member is a magnet 50 if the adaptor 46 is made of metal or has a metal component. That section of the adaptor holder unit 36, which is illustrated on the right of the one-dot, dashed line shown in FIG. 2, is incorporated in the main unit 30. That is, the magnet 50 is provided outside the main unit 30. Near the magnet 50, fitting members 52 are provided, and can fit into the holes or grooves made in the adaptor 46 to set the adaptor 46 at a desired position. The fitting members 52 protrude toward the front of the inverted microscope 12. Therefore, the adaptor 46 is attached to the adaptor holder unit 36 by inserting into the holes or grooves from the front of the inverted microscope 12.

Another magnet 50 and other fitting members 52 may be provided on the back of the adaptor holder unit 36 so that the adaptor 46 may be attached to the adaptor holder unit 36 when the main unit 30 is secured to the left side of the condenser lens 26. Alternatively, the adaptor holder unit 36 may be replaced by another, depending upon the position at which the main unit 30 is secured.

As shown in FIG. 3, the needle 44 attached to the adaptor 46 is composed of a cantilever tip 54 and a shaft 56 holding the cantilever tip 54. The cantilever tip 54 has the tip unit 42 mentioned above. The cantilever tip 54 is adhered to the distal end of the shaft 56.

The cantilever tip 54 has been manufactured by a silicon process and is composed of a silicon base unit 58, a flexible lever unit 60, and the above-mentioned tip unit 42. The silicon base unit 58 is a part to which another part, i.e., the shaft 56 is adhered. The lever unit 60 extends from the silicon base unit 58 and has, for example, thickness of 2.7 μm, length of 240 μm and elastic constant of about 2 N/m. The tip unit 42 is formed at the free end of the lever unit 60, at an angle of about 90° to the lengthwise direction of the lever unit 60.

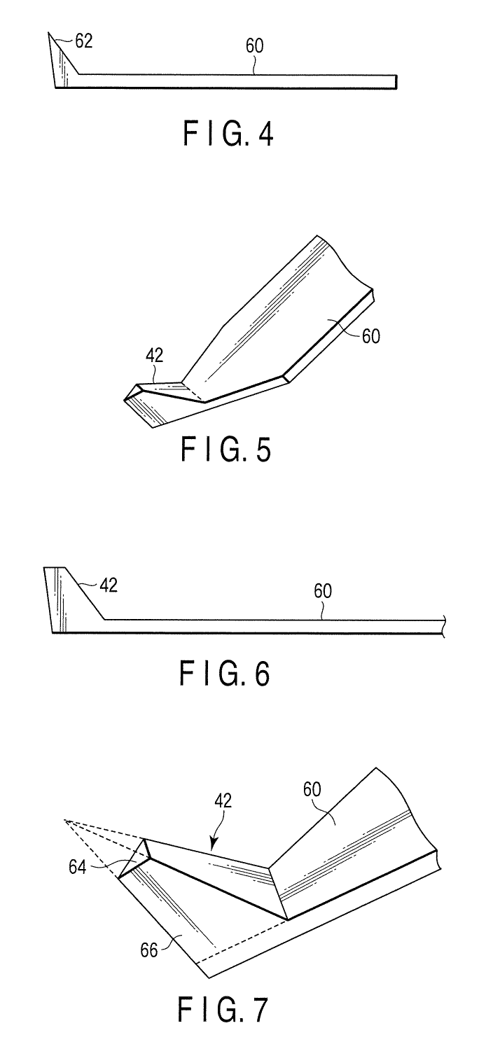

Such a tip unit 62 as shown in FIG. 4, which has a sharpened tip, is used in the tip drive apparatus of the ordinary type. In contrast, in the tip drive apparatus 10 according to this embodiment, the tip unit 42 has a distal end that is flat and extends almost parallel to the lever unit 60 as shown in FIGS. 5 and 6. That is, as shown in FIG. 7, the tip unit 42 has a first region (distal-end surface) 64 and second regions (lateral surfaces) 66. The first region 64 includes a contact edge which lies in the cross section including the distal end of the lever unit 60 and which contacts the cell, applying a prescribed pressure to the cell. The second regions 66 are continuous to the lever unit 60.

In the tip drive apparatus 10 according to this embodiment, the needle 44 incorporating the tip unit 42 is inserted into, and held in, the hole (not shown) made in the adaptor 46. Thereafter, the adaptor 46, now holding the needle 44, is attached to the main unit 30. The needle 44, which is basically an expendable article and replaced frequently, can thus be replaced by a new one. Therefore, the tip drive apparatus 10 can be used over again, without the risk of contamination.

Assume that the needle 44, which is a thin and long member, is directly attached to the main unit 30. Then, the operability decreases, and the tip unit 42 may hit a part, such as microscope XY stage 16, of the inverted microscope 12 during the attaching operation, and may possibly be broken while being attached to the main unit 30. In this embodiment, the needle 44 is first attached to the adaptor 46 removed from the main unit 30, and the adaptor 46 is then secured at the front of the main unit 30. Hence, the risk of damaging the tip unit 42 can be reduced.

The adaptor 46 is configured to hold the shaft 56 of the needle 44, at a prescribed angle and in a downwardly inclined position, when the adapter is attached to the main unit 30. Further, the cantilever tip 54 is adhered to the shaft 56, at a prescribed angle to the shaft 56. Moreover, as pointed out above, the tip unit 42 is provided, extending in a direction to intersecting with the lengthwise direction of the lever unit 60. The tip unit 42 is therefore held with its distal end directed downward, almost in the vertical direction, at the free end of the lever unit 60, as long as the adaptor 46 remains secured to the main unit 30.

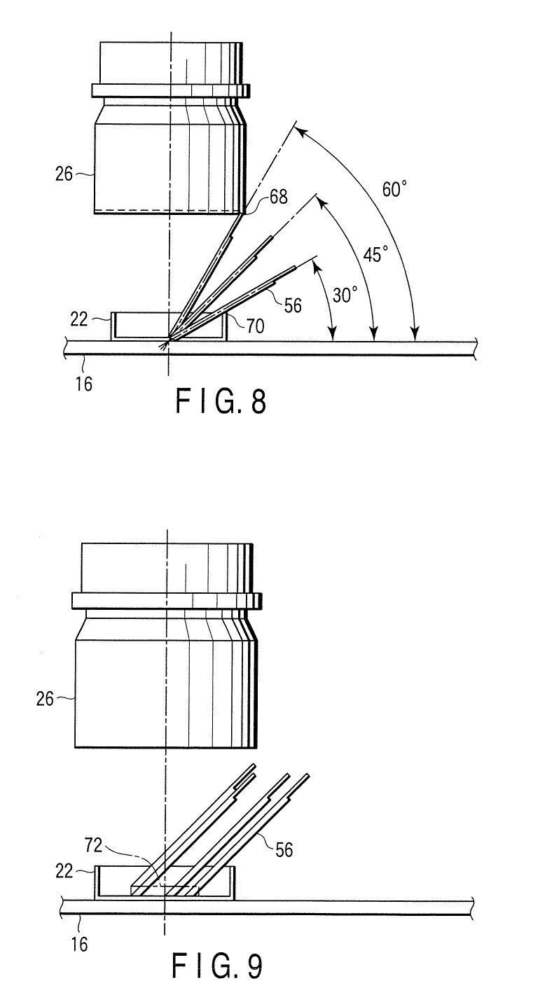

The angle at which the adaptor 46 holds the shaft 56 is determined as will be explained below. If the shaft angle indicated in FIG. 8 is too large, it will inevitably interfere with the condenser lens 26. Assume that the needle 44 is, for example, about 50 mm long. Then, the shaft 56 interferes with the condenser lens 26 if the shaft angle larger is larger than 60°. Conversely, if the shaft angle larger is too small, the shaft 56 inevitably interferes with the sidewall of the dish 22 as indicated by reference number 70 in FIG. 8. The dish 22 may be a glass-bottom of 35 mm dish that is usually used in cell culture. In this case, the shaft 56 interferes with the dish 22 if it is rotated upward by less than 30°. This is why the adaptor 46 holds the shaft 56 at the angle of 45° that is the intermediate value between 30° and 60°.

If the adaptor 46 holds the shaft 56 at the angle of 45°, there will be provided such a movement region 72 as indicated by a one-dot, dashed line shown in FIG. 9. The work can be proceeded, without the risk that the glass surface (having a diameter of about 14 mm) of the glass bottom dish of 35 mm interferes with the condenser lens 26 or the sidewall of the dish 22.

Thus, the angle at which the adaptor 46 holds the shaft 56 is determined to enable the needle 44 to have an sufficient movement region 46, in consideration of possible interference with the condenser lens 26 and the dish 22 used. The adaptor 46 has a hole (not shown) so shaped that the needles 44 may be inserted and held the shaft 56 at the angle so determined.

As shown in FIG. 1, the operation module 34 of the tip drive apparatus 10 has a Z-value adjustment handle 74, a speed setting dial 76, a minute-adjustment (up) button 78, a minute-adjustment (down) button 80, a movement setting dial 82, and a Z-value setting button 84.

The Z-value adjustment handle 74 and speed setting dial 76 are used to move coarsely the adaptor holder unit 36 in the Z-direction (in units of millimeters). As the Z-value adjustment handle 74 is rotated, the Z-drive unit 38 drives the adaptor holder unit 36 in the Z-direction, in accordance with the rotation of the Z-value adjustment handle 74. When operated, the speed setting dial 76 switches the distance by which to move the unit 36 as the Z-value adjustment handle 74 is rotated, from any one of the three values, i.e., long, intermediate and short, to another value.

The minute-adjustment buttons 78 and 80 and movement setting dial 82 are used to move minutely the adaptor holder unit 36 in the Z-direction (in units of microns). As the minute-adjustment (up) button 78 or minute-adjustment (down) button 80 is operated, the Z-drive unit 38 drives the adaptor holder unit 36 minutely in the Z-direction, in accordance with the operation of the button. The movement setting dial 82 switches the distance by which to move the unit 36 as the minute-adjustment button 78 or 80 is operated one time, from any one of the three values, i.e., long, intermediate and short, to another value.

The Z-value setting button 84 is a button, which may be pushed to instruct that any position in the Z-direction be stored. Even if the Z-value adjustment handle 74 or the minute-adjustment button 78 or 80 is operated, the adaptor holder unit 36 will never move down below the position stored by pushing the Z-value setting button 84 (toward the sample placed in the dish 22). The Z-value setting button 84 has a latch mechanism (not shown). Once depressed or turned on by the operator, the Z-value setting button 84 remains in the on state until it is depressed again. Hereinafter, the operation of the Z-value adjustment handle 74 and the minute-adjustment buttons 78 and 80 while the Z-value setting button 84 remains in the off state is called the “first mode,” and the operation of the Z-value adjustment handle 74 and the minute-adjustment buttons 78 and 80 while the Z-value setting button 84 remains in the on state is called the “second mode.”

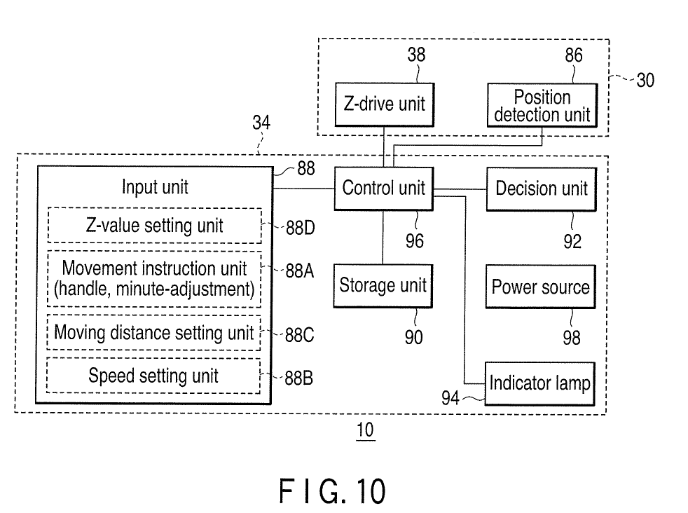

As shown in FIG. 10 illustrating the electrical configuration of the tip drive apparatus 10 according to this embodiment, the main unit 30 has, in addition to the Z-drive unit 38, a position detection unit 86 that is configured to detect the position of the adaptor holder unit 36. The position detection unit 86 may directly detect the position of the adaptor holder unit 36 by using optical means, or may indirectly detect the position of the adaptor holder unit 36 by detecting the distance by which the Z-drive unit 38 has been driven. Furthermore, the position detection unit 86 may be provided as a unit separated from the main unit 30.

The operation module 34 has an input unit 88, a storage unit 90, a decision unit 92, an indicator lamp 94, a control unit 96, and a power source 98.

The input unit 88 includes a movement instruction unit 88A, a speed setting unit 88B, a moving distance setting unit 88C, and a Z-value setting unit 88D. The movement instruction unit 88A outputs a movement instruction when the Z-value adjustment handle 74 is operated and the minute-adjustment button 78 or 80 is turned on. The speed setting unit 88B outputs a speed setting signal representing the moving speed set as the speed setting dial 76 is rotated. The moving distance setting unit 88C outputs a distance setting signal representing the distance set as the movement setting dial 82 is rotated. The Z-value setting unit 88D outputs a Z-value setting signal when the Z-value setting button 84 is turned on. The signals output from the input unit 88 are input to the control unit 96.

The storage unit 90 stores, as the Z-value, the position of the adaptor holder unit 36, which the position detection unit 86 detects when the Z-value setting button 84 is turned on. The decision unit 92 compares the position of the adaptor holder unit 36, which the position detection unit 86 has detected, with the Z-value stored in the storage unit 90, thereby determining whether the adaptor holder unit 36 has reached the position of the Z-value. The indicator lamp 94 blinks in response to the Z-value setting signal output from the Z-value setting unit 88D. Seeing the indicator lamp 94 blinking, the operator can confirm that the Z-value has been duly stored.

The control unit 96 controls the entire tip drive apparatus 10. The power source 98 supplies power to the components of the tip drive apparatus 10.

A tip driving method, which uses the tip drive apparatus 10 according to this embodiment, will be explained below.

Here, a case will be described, in which the tip drive apparatus 10 according to this embodiment is used to introduce a substance into cells being cultured in a culture solution filled in the dish 22.

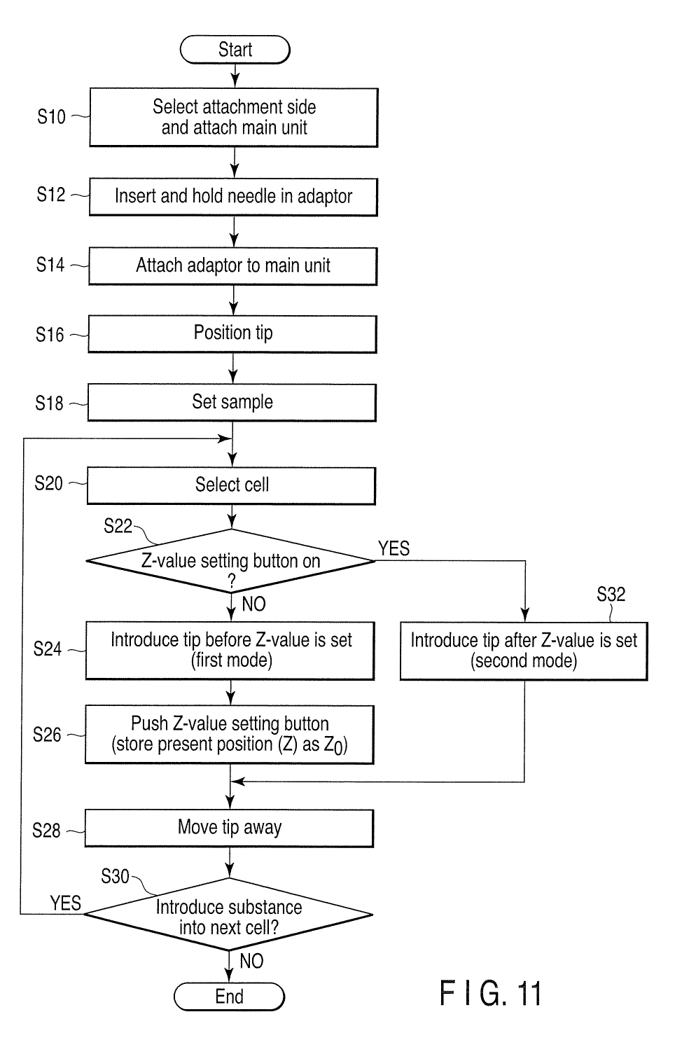

As shown in FIG. 11, the side to which the main unit 30 should be attached is selected, and the main unit 30 is then attached to the condenser lens 26 via the microscope adaptor 32 (Step S10).

Next, the needle 44 is inserted into, and held in, the adaptor 46 removed from the main unit 30 (Step S12). The adaptor 46 holding the needle 44 is attached to the adaptor holder unit 36 of the main unit 30, from the front of the inverted microscope 12 (Step S14).

Thereafter, the tip is positioned (Step S16). That is, while observing the needle 44, the operator brings the tip unit 42 formed at the distal end of the needle, to the center (i.e., view-field center) of the eyepiece (not shown). The operator accomplishes this by manipulating the needle-tip XY adjustment knob 40 of the main unit 30 and the Z-value adjustment handle 74 of the operation module 34. This manipulation is performed, not having the dish 22 mounted on the microscope XY stage 16. As for the Z-direction, the operator turns the speed setting dial 76 of the operation module 34, setting the long or intermediate distance, and then operates the Z-value adjustment handle 74, thereby lowering the tip unit 42 to a position where he or she can see the lever unit 60 of the cantilever tip 54.

When the tip unit 42 is so positioned, a sample is set, more precisely, the dish 22 is mounted on the microscope XY stage 16 (Step S18). This step is performed in the following sequence. First, the Z-value adjustment handle 74 of the operation module 34 is operated, moving the tip unit 42 at the distal end of the needle 44, to a safe region (upward in the Z-direction). An arm 100 (FIG. 1) of the inverted microscope 12 is then pulled back. As a result, the main unit 30 is moved as a whole. A space for sample setting is thereby provided. Then, the dish 22 (sample) is mounted on the microscope XY stage 16. Thereafter, the arm 100 of the inverted microscope 12 is moved to the initial position. Note that the dish 22 (sample) so set contains a substance in a dispersed state, which will be introduced into cells being cultured in a culture solution held in the dish 22.

Next, the cell into which the substance should be introduced is selected (Step S20). First, the operator manipulates the microscope XY stage handle 18, moving the microscope XY stage 16 and, thereby, arranging the cell held in the dish 22 into the view field of the microscope so that the cell may be observed. Thereafter, the operator actuates the Z-drive unit 38, moving the tip unit 42 of the needle 44 toward the cell from above. That is, while observing through the eyepiece 20, the operator lowers the tip unit 42 in the Z-direction until the lever unit 60 of the cantilever tip 54 comes into the view field and become visually confirmed. This is achieved by first turning the speed setting dial 76 of the operation module 34, setting the low speed, and then operating the Z-value adjustment handle 74. Since the cells in the dish 22 are not at the same height as the tip unit 42, the tip unit 42 is not focused and can hardly be observed. Therefore, the operator moves the tip unit 42 down in the Z-direction, using the lever unit 60 as index. This is because the lever unit 60 is larger than the tip unit 42 and therefore the lever unit 60 can be recognized generally, even if its image is not focused. After the lever unit 60 moves to a position where it is visually recognized, the operator adjust the position of the microscope XY stage 16 with respect to X direction and Y direction, while observing the cells through the eyepiece 20. The tip unit 42 is thereby set at a position that seems right above the cell into which to introduce the substance. Thus, the cell into which to introduce the substance is selected.

The following operation depends on whether the Z-value has been set or not in the storage unit 90 of the operation module 34.

When the tip is driven for the first time, the Z-value has yet to be set in the storage unit 90 (Step S22). Therefore, the tip is introduced in the first mode (without using the Z-value) (Step S24). That is, the operator determines an optimal position in the Z-direction, while operating the Z-value adjustment handle 74 or the minute-adjustment button 78 or 80 and observing through the eyepiece 20, confirming “the distortion of the cell” or “the bending of the lever unit 60.” At this point, the Z-value adjustment handle 74 is manipulated, while the speed setting dial 76 is turned, switching the speed from any one of the three values, i.e., high, medium and low, to another value. The minute-adjustment button 78 or button 80 is operated, while the movement setting dial 82 is turned, switching the distance from any one of the three values, i.e., long, intermediate and short, to another value.

As the tip unit 42 is thus moved down toward the bottom of the dish 22, lowering the distal end of the tip unit 42, the tip unit 42 contacts the cell in the dish 22. If the tip unit 42 is further lowered, the distal end of the tip unit 42 will pass through the cell membrane and penetrates the cell nucleus, forming a scar or hole in the membrane and nucleus. The substance dispersed in the dish 22 therefore flows into the cell through the formed scar or hole. The substance may flow into the cell, without forming a scar or hole, depending on the size of particles to introduce, if the channel coupled to a stretch receptor or the like is opened when the tip unit 42 deforms the cell, applying a physical stimulus to the cell. Thus, the substance is introduced.

When the substance is so introduced, the operator may push the Z-value setting button 84 of the operation module 34. Then, the control unit 96 of the operation module 34 determines that the Z-value setting button 84 has been pushed. In this case, the control unit 96 makes the storage unit 90 stores, as the Z-value representing an optimal position, the present position of the adaptor holder unit 36, which the position detection unit 86 has detected (Step S26). At this point, the control unit 96 turns on the indicator lamp 94.

The distal end of the tip unit 42 is made to slide as described above, incising the cell, thereby introducing the substance into the cell.

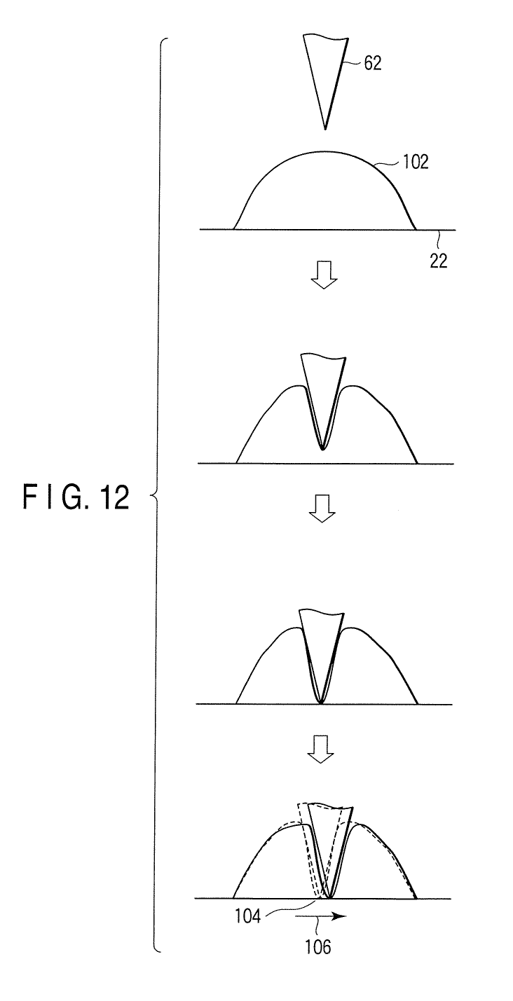

As shown in FIG. 12, a cell 102 will have a scar 104 because of the point contact and the sliding, if the sharpened tip 62 of the conventional tip drive apparatus is used. Being sharp, the distal end of the tip unit 62 will probably be broken as it slides as indicated by arrow 106 shown in FIG. 12.

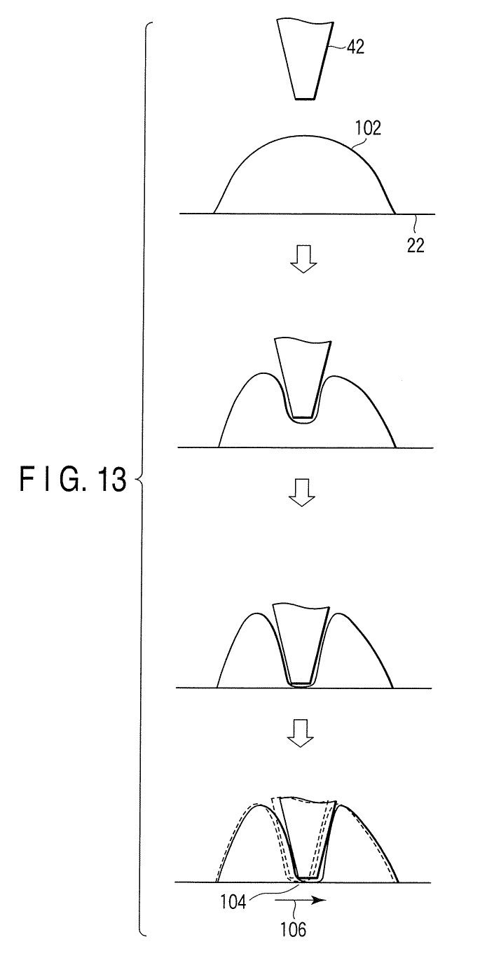

The present embodiment uses the tip unit 42 having a flat distal end as shown in FIG. 13. Not having a sharp distal end, there is little risk of the tip unit 42 being broken when it slides. If the tip unit 42 is set in surface contact with the cell 102, the area at which the tip unit 42 contacts the cell 102 will increase. The tip unit 42 can therefore make a somewhat large scar 104 in the cell 102. This increases the efficiency of introducing the substance into the cell 102. Moreover, the damage to the cell 102 can be reduced if the tip unit 42 contacts the cell 102, only at its corner. Further, the present embodiment can easily control the amount of substance introduced in an optimal way, in two regions 64 and 66, whereas the conventional tip unit 62 controls the amount in the second region 66 only because it does not have a region equivalent to the first region 64. This helps to stabilize the amount of substance introduced into the cell.

The ratio of the first region 64 to the second region 66 in terms of area may be determined to introduce the substance in an optimal amount.

Thereafter, the operator operates the Z-value adjustment handle 74 of the operation module 34, raising the needle 44 and, thus, moving up the tip unit 42 (Step S28). That is, the operator turns the speed setting dial 76 of the operation module 34, switching the speed to the medium speed or the low speed, and then operates the Z-value adjustment handle 74 at the first mode (no Z-value), raising the tip unit 42.

After the tip unit 42 is raised and pulled from the cell 102, and after a certain time has passed, the cell membrane is restored by itself and now contains the substance.

Assume that the tip unit 42 has been completely moved up (Step S28). Then, whether the substance should be introduced into the next sample cell is determined (Step S30). If NO, the operator turns off the power switch (not shown) of the main unit 30, terminating the tip driving method.

On the other hand, if the substance should be introduced into any other cell (Step S30), the method returns to Step S20. Thus, the substance may be introduced into other sample cells, one after another. That is, the operator manipulates the microscope XY stage handle 18, while making observation through the eyepiece 20, thereby actuating the microscope XY stage 16 and setting the tip unit 42 right above the cell 102 into which to introduce the substance. In other words, the operator selects the cell 102 into which the substance should be introduced (Step S20).

At the time the tip is driven for the second time, and so forth, the Z-value has already been set in the storage unit 90 (Step S22). Therefore, the tip is introduced in the second mode (by using the Z-value) (Step S32). In this case, the Z-value has been set in the storage unit 90. Therefore, the operator can lower the tip unit 42 to an optimal position, merely by fully lowering the tip unit 42, without worrying about an excessive manipulation of the Z-value adjustment handle 74 and minute-adjustment buttons 78 and 80, once the tip unit 42 has been positioned in the horizontal direction. That is, the decision unit 92 of the operation module 34 compares the position of the adaptor holder unit 36, detected by the position detection unit 86, with the Z-value set in the storage unit 90, determining whether the adaptor holder unit 36 (tip unit 42) has reached the position of the Z-value. If the decision unit 92 determines that the adaptor holder unit 36 is found to have reached this position, the control unit 96 of the operation module 34 controls the Z-drive unit 38, preventing the same from moving down further, even if the Z-value adjustment handle 74 and the minute-adjustment buttons 78 and 80 are operated.

Since the optimal Z-position is set in the storage unit 90, the adaptor holder unit 36 (tip unit 42) may be automatically lowered to the optimal Z-position. That is, the handle operation in the second mode may be automated.

If the inverted microscope 12 is an electrically-driven type, a computer controls and drives the microscope XY stage 16. In this case, the inverted microscope 12 has a CCD camera or the like, and can display images on a monitor. If the inverted microscope 12 is such an electrically-driven type, not a manually operable type, any cell into which the substance should be introduced may be selected on the monitor screen, and the tip unit may be automatically moved to its position. In other words, the microscope XY stage 16 may be automatically adjusted in the XY direction.

Note that the substance to introduce into the cell may be anything that can be dispersed in the dish 22, such as genes, dyes, fluorescent reagent, e.g., quantum dots, ions, peptides, proteins or polysaccharides.

The cross-sectional shape of the flat surface (first region 64), which contacts the cell 102, is not limited to such a triangular one as shown in FIGS. 5 and 7. Rather, it may be square or have more sides (i.e., polygonal). That is, the first region 64 may have any shape so long as it is not a point or a tube.

### EXAMPLES

A first example is HelaS3 cells which were immersed in a gene solution, and into which genes were introduced. The genes express GFP fluorescent protein. Whether the genes were successfully introduced or not can be confirmed by performing fluorescence observation.

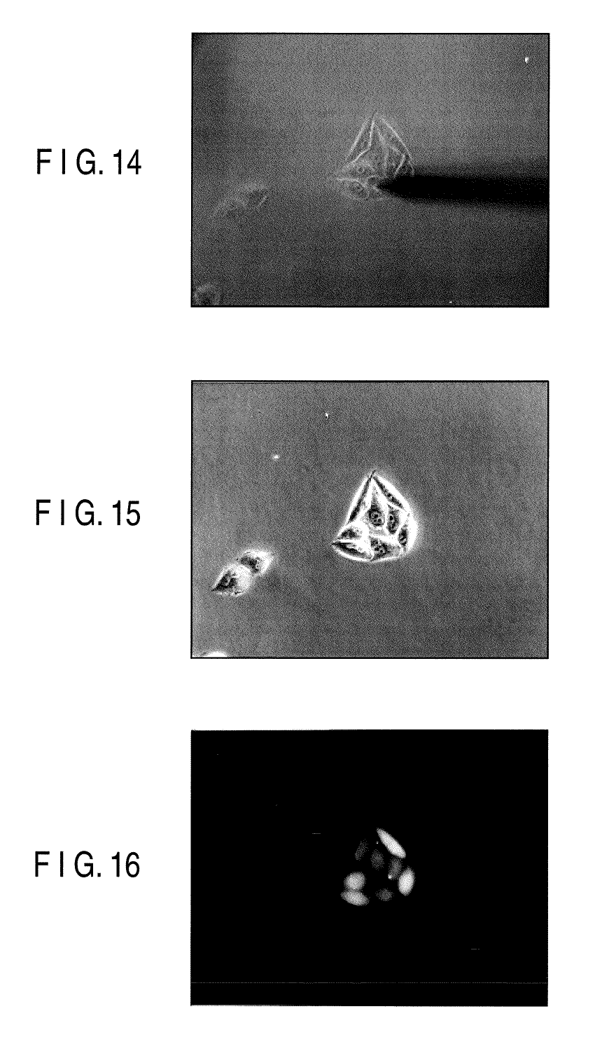

FIG. 14 presents a microscope image of the cells, obtained immediately after genes had been introduced. FIG. 15 and FIG. 16 are microscope images of the cells, acquired 24 hours after introducing the genes, which show whether the genes have been introduced into the cells. The image of FIG. 15 is one observed through a phase contrast microscope, showing the state the cells had 24 hours after the genes had been introduced. FIG. 16 is one obtained through fluorescence observation. In the cell into which the genes had been successfully introduced, the genes well expressed, attaining intense fluorescent light. This proves that the genes were introduced into the cells at a very high efficiency.

As described above, the tip drive apparatus 10 according to this embodiment uses the tip unit 42 that has the first region 64 including one side at which a prescribed pressure is applied to the cell 102 in a cross section including the distal end of the lever unit 60 and extending along the lever unit 60. This stabilizes the amount of substance introduced into the cell. Hence, this embodiment can introduce the substance into the cell not only in such a low-invasive manner while maintaining such a high cellular survival rate as the conventional tip drive apparatus, but also with high reliability and high efficiency.

### Second Embodiment

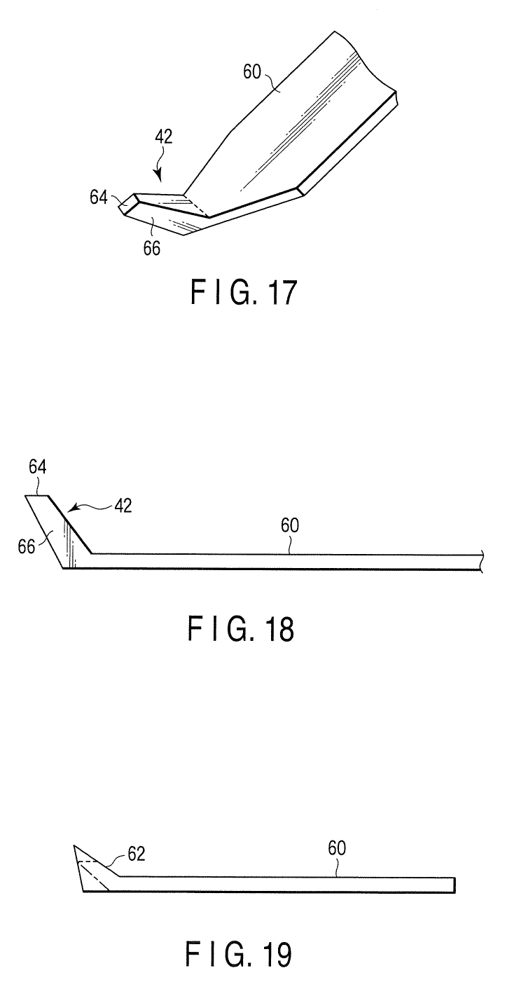

A tip drive apparatus according to a second embodiment of this invention has a tip unit 42 including the second region 66 which is smaller in area than in the first embodiment, as shown in FIGS. 17 and 18. The first region 64 is square as shown in FIG. 17. It may instead be triangular or have more sides (i.e., polygonal), as in the first embodiment described above.

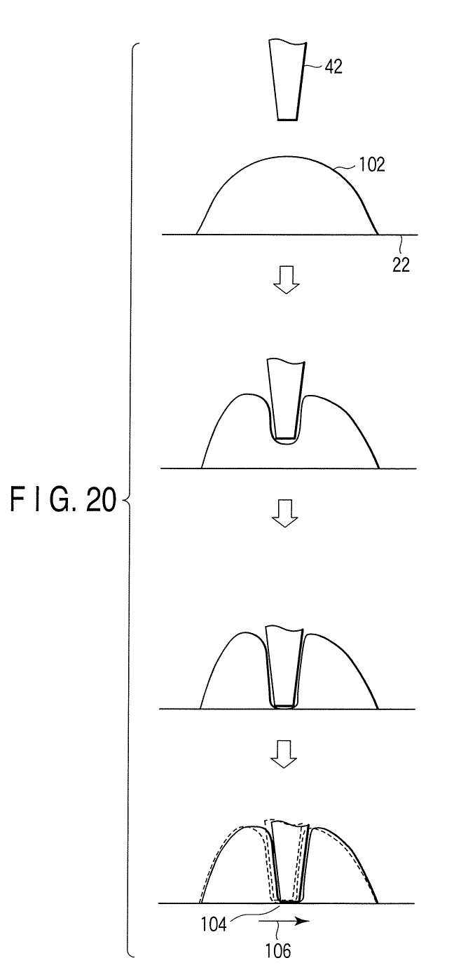

The tip unit 42 according to this embodiment can be produced by cutting the commercially available cantilever tip that has, for example, such a sharp tip unit 62 as shown in FIG. 19. That is, the commercially available cantilever tip having the sharp tip unit 62 may be cut in two directions, along the one-dot dashed line and the broken line, both shown in FIG. 19.

Alternatively, the tip unit 42 according to the first embodiment, which has been made through a silicon process, may be cut in one direction, along the one-dot dashed line shown in FIG. 19.

As has been explained in conjunction with the first embodiment, the ratio between the first region 64 and second region 66 of the tip unit 42, in terms of area, may be determined to introduce the substance in an optimal amount. In order to set such a ratio, the commercially available cantilever tip having the sharp tip unit 62 may be cut in two directions. Alternatively, a cantilever tip 54 that has a tip unit 42 having the first and second regions 64 and 66, as in the first embodiment, may be cut in one direction.

The distal end of the tip unit 42 of this embodiment can be made to slide as indicated by arrow 106 shown in FIG. 20, incising the cell, thereby introducing the substance into the cell, in the same way as the distal end of the tip unit 42 of the first embodiment, which is shown in FIG. 13. In the present embodiment, the second region 66 of the tip unit 42 is smaller than in the first embodiment. The amount of substance introduced, which is proportional to the volume of the tip unit 42, is therefore smaller than in the first embodiment. The load the tip unit 42 exerts on the cell 102 is, however, smaller than in the first embodiment.

As indicated above, also in the tip drive apparatus 10 according to the second embodiment, the amount of substance introduced into the cell can be stabilized. Hence, the second embodiment can introduce the substance into the cell not only in such a low-invasive manner while maintaining such a high cellular survival rate as the conventional tip drive apparatus, but also with high reliability and high efficiency.

Moreover, the load exerted on the cell 102 is small. Therefore, the tip drive apparatus is suitable for introducing the tip into cells more weakly (smaller or slimmer) than the cells into which the tip unit 42 of the first embodiment may be inserted.

### Third Embodiment

In the first and second embodiments described above, one tip drive apparatus 10 is secured to the inverted microscope 12 and is used. A plurality of tip drive apparatuses 10 may be used at the same time. For example, main units 30 may be secured to the sides of the condenser lens 26.

If a plurality of tip drive apparatuses 10 are used in this manner, tips can be used not only to introduce substances, but also to, for example, apply an electric signal between the tip units 42, thereby to give cells an electrical stimulus. The electrical stimulus is not necessarily be given exclusively by using a plurality of tip units 42. Rather, it can be given by applying a potential difference between a tip unit 42 and a particular electrode (e.g., glass bottom with ITO). If this is the case, the tip unit 42 had better be electrically conductive.

Thus, this embodiment can apply an electrical stimulus to cells, not only in such a low-invasive manner and maintaining the cell at a high survival rate, enabling the operator to observe the living cells with high efficiency.

This invention has been explained, with reference to various embodiments. The invention is not limited to the embodiments described above, nevertheless. Various changes and modifications can, of course, be made within the scope of this invention.

For example, the microscope adaptor 32 extending from the main unit 30 of the tip drive apparatuses 10 may be attached to a support unit supporting the condenser lens 26, not to the condenser lens 26 as in the embodiments described above.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A tip drive apparatus capable to moving a tip unit toward an object, while holding the tip unit at a prescribed angle, the tip unit being formed on a support unit having flexibility and directed to the object at the prescribed angle, the tip unit includes:
a contact side in a cross section including the distal end of the support unit and extending in the direction the support unit extends, the contact side configured to contact, at one part at least, the object, and to apply a prescribed pressure to the object;
a first region including the contact side; and
a second region continuous to the support unit.

2. The tip drive apparatus according to claim 1, wherein the first region is flat surface.

3. The tip drive apparatus according to claim 1, wherein the contact side is almost parallel to the support unit.

4. The tip drive apparatus according to claim 1, wherein an area ratio between the first region and second region of the tip unit is determined to introduce the tip unit by an optimal distance.

5. A cantilever tip comprising:
a support unit having flexibility; and
a tip unit formed at a prescribed angle to the support unit, and configured to be attached, via a particular member, to a tip drive apparatus capable of moving the tip unit in a predetermined direction,
wherein the tip unit includes:
a contact side in a cross section including the distal end of the support unit and extending in the direction the support unit extends, the contact side configured to contact, at one part at least, an object, and to apply a prescribed pressure to the object;
a first region including the contact side; and
a second region continuous to the support unit.
